(19) United States
(12) Patent Application Publication
(10) Pub. No.: US 2019/0030357 A1
(43) Pub. Date: Jan. 31, 2019

(54) TRANSCRANIAL MAGNETIC STIMULATION CIRCUIT, TRANSCRANIAL MAGNETIC STIMULATOR AND MAGNETIC IMPULSE GENERATION METHOD THEREOF

(71) Applicant: SHENZHEN SHENGLI TECHNOLOGY CO., LTD, SHENZHEN CITY (CN)

(72) Inventor: FEI YUE XIE, YUEYANG CITY (CN)

(21) Appl. No.: 15/663,906

(22) Filed: Jul. 31, 2017

(51) Int. Cl. A61N 2/02; A61N 2/00

(52) U.S. Cl. CPC A61N 2/02; A61N 2/006

(57) ABSTRACT

A transcranial magnetic stimulation circuit, a transcranial magnetic stimulator and a magnetic impulse generation method thereof are provided, in which the circuit includes at least two inductance coils, a direct current, a charging and discharging circuit and a controller. The controller controls the inductance coils to synchronously generate magnetic impulses through an electronic switch. The method includes: coinciding central lines of the inductance coils with each other; controller outputting an impulse sequence to control the electronic switch to enable the inductance coils to synchronously generate magnetic impulses; and superposing the magnetic impulses to form the magnetic impulses required by the transcranial magnetic stimulator. The transcranial magnetic stimulator includes a pillow base and the transcranial magnetic stimulation circuit. A containing cavity is disposed in the pillow base. The inductance coils are disposed in the containing cavity and their central lines of the inductance coils are coincided with each other.

### BACKGROUND

### 1. Technical Field

The present disclosure relates to a transcranial magnetic stimulation, in particular, to a transcranial magnetic stimulation circuit, a transcranial magnetic stimulator and a magnetic impulse generation method thereof.

### 2. Description of Related Art

The fundamental principle of the transcranial magnetic stimulation (TMS) is to power up the impulse current in the inductance coil to generate a pulsed magnetic field. As shown in FIG. 1, a conventional transcranial magnetic stimulator is to use a general RCL circuit structure, in which R is resistance, L is inductance, C is capacitance, and is charged by alternating current. The working principle is as follows: boosting and filtering the alternating current, turning off the control switch K1, disconnecting the control switch K2, and rapidly charging the inductance C; then disconnecting the control switch K1, turning off the control switch K2, discharging the capacitance C, and using the oscillating circuit LC to enable the current in the inductance coil L to have a fast change to generate a pulsed magnetic field. An impulse width of an impulse depends on the oscillation period T of the LC circuit (T=2\sqrt{LC}).

The formula of inductive energy of the inductance coil is J_{L}=0.5×L×I². In order to increase the magnetic induction strength of the magnetic impulse generated by the inductance coil L (generally referring to the magnetic induction strength at one point on the central axis of an inductance coil), the charging voltage of the capacitance C has to be increased so as to enable the inductance coil L to obtain a larger impulse current. However, excessive current would cause the technical problems concerning that the inductance coil is susceptible to heating, the insulation strength of the coil is difficult to be improved, the charge time of the capacitance C becomes longer, the pressure resistance of the control switch has to be increased, the line loss of the circuit increases, and so on. However, the design and fabrication scheme of a product would take those factors mentioned above into consideration, which may result in complicated structure, bulky volume, and higher production cost and usage cost, much less the difficulty in miniaturizing the product.

### SUMMARY

The primary purpose of the present disclosure is to provide a transcranial magnetic stimulation circuit, a transcranial magnetic stimulator and a magnetic impulse generation method thereof to overcome the above-mentioned drawbacks.

According to one exemplary embodiment of the present disclosure, a transcranial magnetic stimulation circuit is provided, including at least two inductance coils; a direct current, connected to the at least two inductance coils through an electronic switch to form a charging circuit of the at least two inductance coils; a discharging circuit of the at least two inductance coils; and a controller, connected with a control end of the electronic switch and configured to control the at least two inductance coils to synchronously generate magnetic impulses.

In a preferred embodiment, the transcranial magnetic stimulation circuit further comprises: first resistances of which an amount is equal to the at least two inductance coils, wherein the first resistances and the at least two inductance coils are in series connection for adjusting charging and discharging time constants of the at least two inductance coils to ensure that the at least two inductance coils synchronously generate magnetic impulses.

In a preferred embodiment, the discharging circuit comprises a series branch formed by a second resistance and a diode.

According to another exemplary embodiment of the present disclosure, a magnetic impulse generation method for a transcranial magnetic stimulator is provided, wherein a transcranial magnetic stimulation circuit of the transcranial magnetic stimulator comprises: at least two inductance coils; a direct current, connected to the at least two inductance coils through an electronic switch to form a charging circuit of the at least two inductance coils; a discharging circuit of the at least two inductance coils; and a controller, connected with a control end of the electronic switch and configured to control the at least two inductance coils to synchronously generate magnetic impulses. The magnetic impulse generation method comprises: coinciding central lines of the at least two inductance coils with each other; and the controller outputting an impulse sequence to control the electronic switch to enable the at least two inductance coils to synchronously generate magnetic impulses, and superposing the synchronously generated magnetic impulses to form the magnetic impulses required by the transcranial magnetic stimulator.

In a preferred embodiment, the magnetic impulse generation method further comprises: the controller controlling an impulse frequency of the outputted magnetic impulses according to a parameter inputted by a user to control a generation frequency of magnetic impulses.

In a preferred embodiment, the magnetic impulse generation method further comprises: the controller controlling an impulse width of the outputted magnetic impulses according to a parameter inputted by a user to control the magnetic impulse strength.

In a preferred embodiment, the magnetic impulse generation method further comprises: the controller controlling an impulse frequency of the outputted magnetic impulses according to a parameter inputted by a user to control a generation frequency of magnetic impulses; and the controller controlling an impulse width of the outputted magnetic impulses according to a parameter inputted by a user to control the magnetic impulse strength.

In a preferred embodiment, in the magnetic impulse generation method for the transcranial magnetic stimulator circuit, the transcranial magnetic stimulator circuit of the transcranial magnetic stimulator further comprises: first resistances of which an amount is equal to the at least two inductance coils, wherein the first resistances and the at least two inductance coils are in series connection for adjusting charging and discharging time constants of the at least two inductance coils to ensure that the at least two inductance coils synchronously generate magnetic impulses.

According to yet another exemplary embodiment of the present disclosure, a transcranial magnetic stimulator is provided, comprising: a transcranial magnetic stimulation circuit and a pillow base, wherein the transcranial magnetic stimulation circuit includes: at least two inductance coils; a direct current, connected to the at least two inductance coils through an electronic switch to form a charging circuit of the at least two inductance coils; a discharging circuit of the at least two inductance coils; a controller, connected with a control end of the electronic switch and configured to control the at least two inductance coils to synchronously generate magnetic impulses; a containing cavity provided in the pillow base; wherein when the pillow base is used, the containing cavity is relative to the sleep-induction regions of the brain of a user, the at least two inductance coils of the transcranial magnetic stimulation circuit are disposed in the containing cavity of the pillow base and their central lines are coincided with each other, and a buffer cushion is provided on the at least two inductance coils in the containing cavity.

In a preferred embodiment, the at least two inductance coils of the transcranial magnetic stimulation circuit of the transcranial magnetic stimulator are concentric.

To sum up, compared with its conventional counterparts the present disclosure is advantageous in that a plurality of coils are used to synchronously generate magnetic impulses and then the synchro coupling technology is introduce to stimulate the magnetic impulses, such that the impulse current required by one inductance coil can be greatly decreased, thereby avoiding the technical problems concerning that the inductance coil is susceptible to heating, the insulation strength of the coil is difficult to be improved, the charge time of the capacitance C becomes longer, the pressure resistance of the control switch has to be increased, the line loss of the circuit increases, and so on. In addition, transcranial magnetic stimulators can be miniaturized by virtue of the circuit structure provided by the present disclosure.

### BRIEF DESCRIPTION OF THE DRAWINGS

FIG. 1 is a circuit diagram of a conventional transcranial magnetic stimulator.

FIG. 2 is a schematic diagram of the transcranial magnetic stimulation circuit according to an embodiment of the present disclosure.

FIG. 3 is a schematic diagram illustrating the structure of the transcranial magnetic stimulator according to an embodiment of the present disclosure.

FIG. 4 is an explosion graph of the transcranial magnetic stimulator of FIG. 3.

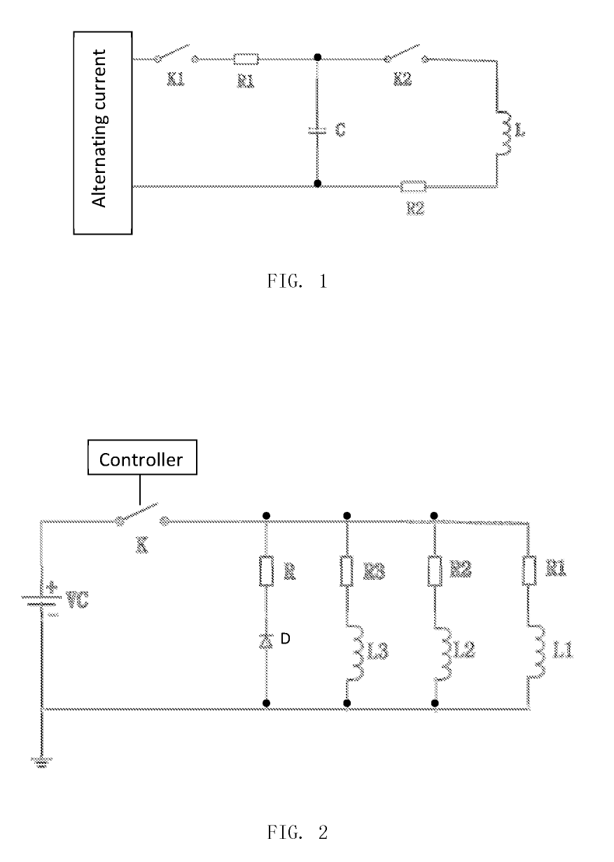

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

Reference is made to FIG. 2. A transcranial magnetic stimulation circuit includes three inductance coils L1, L2, L3, a direct current VC, a discharging circuit of the three inductance coils L1, L2, L3, and a controller. The direct current VC is connected with the three inductance coils L1, L2, L3 through an electronic switch K to form the discharging circuit of the three inductance coils L1, L2, L3. The controller is connected to a control end of the electronic switch K to control the three inductance coils L1, L2, L3 to synchronously generate magnetic impulses. The discharging circuit includes a series branch formed by a second resistance R and a diode D which are in series connection.

A magnetic impulse generation method for the above-mentioned transcranial magnetic stimulation circuit includes the following steps: coinciding central lines of the three inductance coils L1, L2, L3 with each other; the controller outputting an impulse sequence to control the electronic switch K to enable the three inductance coils L1, L2, L3 to synchronously generate magnetic impulses; and superposing the synchronously generated magnetic impulses to form the magnetic impulses required by a transcranial magnetic stimulator. The generation method further includes: the controller controlling an impulse frequency of the outputted magnetic impulses according to a parameter inputted by a user to control a generation frequency of magnetic impulses. In addition, the generation method includes: the controller controlling an impulse width of the outputted magnetic impulses according to a parameter inputted by a user to control magnetic impulse strength.

Because the inductance coil in the conventional RLC circuit structure needs a large impulse current to generate the magnetic impulse having high strength, the transcranial magnetic stimulation circuit provided by the present disclosure employs the following two technical solutions to overcome the drawbacks. 1. RL circuit structure. A smaller impulse current is used to enable an inductance coil to generate magnetic impulse having certain strength. 2. Synchro coupling technology. A plurality of concentric inductance coils (or central lines are coincided with each other) synchronously generate magnetic impulses, and the magnetic impulses are superposed according to the magnetic induction strength vector superposition principle to enable the magnetic impulse strength of the superposed magnetic impulses to reach a desired value at any point on the central axis of the inductance coil. Reference is made to FIG. 2 again. In the embodiment, when the three inductance coils L1, L2, L3 are charged with the same current and their magnetic induction strength generated at the point H on the central axis is respectively denoted as B₁, B₂, B₃, the magnetic induction strength at the point H is B=B₁+B₂+B₃ after synchro coupling. After performing the synchro coupling technology, the impulse current required by one inductance coil can be reduced greatly, so that the technical problems concerning that the inductance coil is susceptible to heating, the insulation strength of the coil is difficult to be improved, the charge time of the capacitance becomes longer, the pressure resistance of the control switch has to be increased, the line loss of the circuit increases, and so on can be effectively avoided. In addition, transcranial magnetic stimulators can be miniaturized by virtue of the circuit structure provided by the present disclosure. It should be noted that those skilled in the art would understand that even though three inductance coils are used in the embodiment in FIG. 2, when performing the synchro coupling technology, the number of inductance coils can be, but not limited to, two or more according to actual requirements.

In the embodiment, for ensuring the three inductance coils L1, L2, L3 to synchronously generate magnetic impulses, the transcranial magnetic stimulation circuit further includes three first resistances R1, R2 R3, in which the three first resistances R1, R2 R3 and the three inductance coils L1, L2, L3 are in serial connection correspondingly. By means of the resistance values of the three first resistances R1, R2 R3, the charging and discharging time constants of the three inductance coils L1, L2, L3 can be substantially similar to each other, thereby ensuring that magnetic impulses can be synchronously generated.

By cooperating the aforementioned transcranial magnetic stimulation circuit and the magnetic impulse generation method with the TMS coupling technology, the technical problems caused by one inductance coil in a conventional transcranial magnetic stimulator requiring a large impulse current can be effectively resolved. In addition, the transcranial magnetic stimulation circuit and the magnetic impulse generation method provided by the present disclosure can simplify the equipment structure, reduce power consumption and production cost, and can be widely used to manufacture various miniaturized transcranial magnetic stimulators.

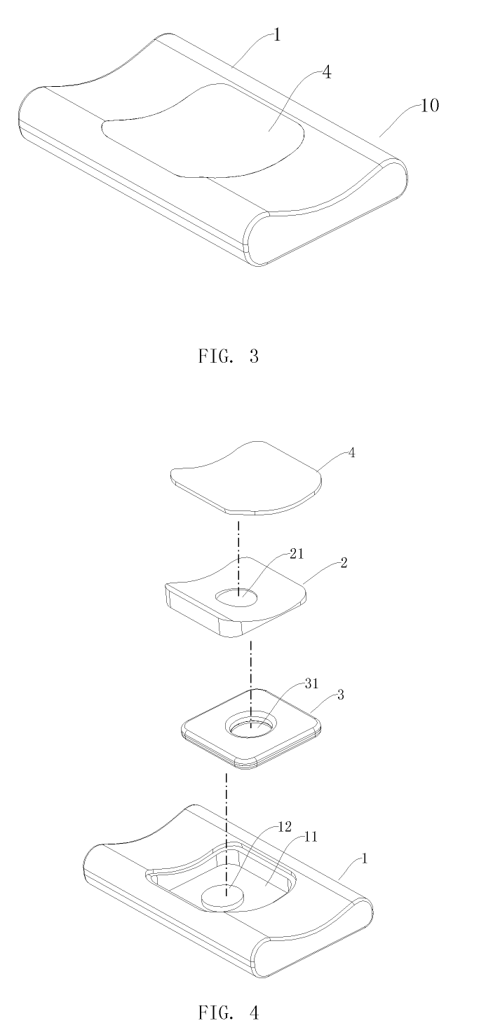

FIG. 3 and FIG. 4 show the structure of the transcranial magnetic stimulator according to an embodiment of the present disclosure. As shown in the figures, the transcranial magnetic stimulator includes a pillow base 1 and a transcranial magnetic stimulation circuit, in which the transcranial magnetic stimulator can employ any transcranial magnetic stimulation circuit as mentioned above. The pillow base 1 is provided with a containing cavity 11. When the pillow base 1 is used, the containing cavity 11 is relative to the sleep-induction regions of the brain of a user. All the inductance coils of the transcranial magnetic stimulation circuit are disposed in the containing cavity 11 of the pillow base 1, and all the inductance coils are concentric. A buffer cushion 2 is provided on the inductance coils in the containing cavity 11, and is used to buffer the contact force between the user's head and the inductance coils. The buffer cushion 2 is also used to ensure the comfortability when the user pillows his/her head on the pillow base 1. In addition, a protection pad 4 is provided on the buffer cushion 2.

All the inductance coils of the transcranial magnetic stimulation circuit are disposed in a casing 3, and a through hole 31 is provided in the middle of the casing 3. Central lines of all the inductance coils of the transcranial magnetic stimulation circuit are coincided with the central line of the through hole 31 of the casing 3. A positioning convex part 12 is provided in the containing cavity 11 of the pillow base 1. The through hole 31 of the casing 3 and the positioning convex part 12 of the containing cavity 11 are engaged with each other. A magnetic hole 21 is provided on the buffer cushion 2.

In a preferred embodiment, the pillow base 1 is made of memory foam or an emulsion material, the buffer cushion 2 is made of memory foam or an emulsion material, and the protection pad 4 is made of a gel.

The above-mentioned descriptions represent merely the exemplary embodiment of the present disclosure, without any intention to limit the scope of the present disclosure thereto. Various equivalent changes, alterations or modifications based on the claims of the present disclosure are all consequently viewed as being embraced by the scope of the present disclosure.

What is claimed is:

1. A transcranial magnetic stimulation circuit, comprising:
at least two inductance coils;
a direct current, connected to the at least two inductance coils through an electronic switch to form a charging circuit of the at least two inductance coils;
a discharging circuit of the at least two inductance coils; and
a controller, connected with a control end of the electronic switch and configured to control the at least two inductance coils to synchronously generate magnetic impulses.

2. The transcranial magnetic stimulation circuit of claim 1, further comprising first resistances of which an amount is equal to the at least two inductance coils, wherein the first resistances and the at least two inductance coils are in series connection for adjusting charging and discharging time constants of the at least two inductance coils to ensure that the at least two inductance coils synchronously generate magnetic impulses.

3. The transcranial magnetic stimulation circuit of claim 1, wherein the discharging circuit comprises a series branch formed by a second resistance and a diode.

4. A magnetic impulse generation method for a transcranial magnetic stimulator, wherein a transcranial magnetic stimulation circuit of the transcranial magnetic stimulator comprises:
at least two inductance coils;
a direct current, connected to the at least two inductance coils through an electronic switch to form a charging circuit of the at least two inductance coils;
a discharging circuit of the at least two inductance coils; and
a controller, connected with a control end of the electronic switch and configured to control the at least two inductance coils to synchronously generate magnetic impulses;
the magnetic impulse generation method comprising:
coinciding central lines of the at least two inductance coils with each other; and
the controller outputting an impulse sequence to control the electronic switch to enable the at least two inductance coils to synchronously generate magnetic impulses, and superposing the synchronously generated magnetic impulses to form the magnetic impulses required by the transcranial magnetic stimulator.

5. The magnetic impulse generation method of claim 4, further comprising: the controller controlling an impulse frequency of the outputted magnetic impulses according to a parameter inputted by a user to control a generation frequency of magnetic impulses.

6. The magnetic impulse generation method of claim 4, further comprising: the controller controlling an impulse width of the outputted magnetic impulses according to a parameter inputted by a user to control the magnetic impulse strength.

7. The magnetic impulse generation method of claim 4, further comprising: the controller controlling an impulse frequency of the outputted magnetic impulses according to a parameter inputted by a user to control a generation frequency of magnetic impulses; and the controller controlling an impulse width of the outputted magnetic impulses according to a parameter inputted by a user to control the magnetic impulse strength.

8. The magnetic impulse generation method of claim 4, wherein the transcranial magnetic stimulator circuit for the transcranial magnetic stimulator further comprises: first resistances of which an amount is equal to the at least two inductance coils, wherein the first resistances and the at least two inductance coils are in series connection for adjusting charging and discharging time constants of the at least two inductance coils to ensure that the at least two inductance coils synchronously generate magnetic impulses.

9. A transcranial magnetic stimulator, comprising:
a transcranial magnetic stimulation circuit, including:
at least two inductance coils;
a direct current, connected to the at least two inductance coils through an electronic switch to form a charging circuit of the at least two inductance coils;
a discharging circuit of the at least two inductance coils;
a controller, connected with a control end of the electronic switch and configured to control the at least two inductance coils to synchronously generate magnetic impulses;
a pillow base, and a containing cavity provided in the pillow base; wherein when the pillow base is used, the containing cavity is relative to the sleep-induction regions of the brain of a user, the at least two inductance coils of the transcranial magnetic stimulation circuit are disposed in the containing cavity of the pillow base and central lines of the at least two inductance coils are coincided with each other, and a buffer cushion is provided on the at least two inductance coils in the containing cavity.

10. The transcranial magnetic stimulator of claim 9, wherein the at least two inductance coils of the transcranial magnetic stimulation circuit are concentric.
